(19) United States
(12) Patent Application Publication
(10) Pub. No.: US 2026/0191828 A1
(43) Pub. Date: Jul. 9, 2026

(54) METHODS AND COMPOSITIONS

(71) Applicant: Sparrow Pharmaceuticals, Inc., Portland, OR (US)

(72) Inventor: David A. Katz, Portland, OR (US)

(21) Appl. No.: 19/441,453

(22) Filed: Jan. 6, 2026

Related U.S. Application Data
(60) Provisional application No. 63/742,054, filed on Jan. 6, 2025.

(51) Int. Cl. A61K 31/4196; A61K 38/26; A61K 47/64; A61K 47/68; A61P 3/04; A61P 3/10; A61P 21/00

(52) U.S. Cl. CPC A61K 31/4196; A61K 38/26; A61K 47/643; A61K 47/6811; A61P 3/04; A61P 3/10; A61P 21/00

(57) ABSTRACT

Provided is a method of administering a GLP-1 receptor agonist to a subject in need thereof, comprising administering an effective amount of the GLP-1 receptor agonist and administering simultaneously, separately, or sequentially in combination an effective amount of a HSD-1 inhibitor.

### CROSS-REFERENCE TO RELATED APPLICATIONS

This application claims priority to U.S. Provisional Application No. 63/742,054, filed Jan. 6, 2025, which is incorporated by reference herein.

### BACKGROUND

Obesity is the most prevalent chronic disease worldwide, affecting as many as 650 million adults. Obesity is associated with numerous complications including cardiovascular disease and type 2 diabetes mellitus (T2DM or T2D), thereby posing a substantial health and economic burden on patients and society.

Glucagon-like peptide 1 (GLP-1) is a 30 amino add long incretin hormone secreted by the L-cells in the intestine in response to ingestion of food. GLP-1 has been shown to stimulate insulin secretion in a physiological and glucose-dependent manner, decrease glucagon secretion, inhibit gastric emptying, decrease appetite, and stimulate proliferation of beta-cells.

GLP-1 receptor agonists attain glycaemic control through mechanisms such as increasing insulin secretion induced by hyperglycemia, inhibiting glucagon secretion during hyperglycemia, slowing gastric emptying, preventing substantial increases in postprandial glucose, and reducing caloric intake and body weight. Short acting GLP-1 receptor agonists (e.g., exenatide and lixisenatide) reduce the effect of nocturnal and fasting glucose but maintain the effect on gastric emptying during long term treatment. Long acting GLP-1 receptor agonists (e.g., liraglutide, exenatide, and dulaglutide) have more profound effects on nocturnal and fasting glucose and glycated haemoglobin (HbA_{1c}), both in the context of oral hypoglycaemic drugs and in combination with basal insulin.

GLP-1 receptor agonists have been shown to greatly reduce the body weight of obese people with type 2 diabetes. The US Food and Drug Administration has approved several different formulations or doses of GLP-1 receptor agonists for the treatment of type 2 diabetes: exenatide, liraglutide, dulaglutide, albiglutide, lixisenatide, semaglutide, and tirzepatide.

However, recent research on GLP-1 receptor agonists for weight loss indicates these medications may lead to significant reductions in lean body mass, including muscle. The STEP 1 and SUSTAIN 8 of semaglutide trials found 39-40% of weight lost on the GLP-1 drugs was lean mass. A 2021 meta-analysis of 18 randomized controlled studies showed significant drops in fat-free mass with GLP-1 drugs, including oral and subcutaneous semaglutide and older GLP-1 drugs such as lixisenatide, exenatide, and liraglutide.

Methods are presently needed to implement treatment with GLP-1 receptor agonists while preventing muscle loss and inducing preservation of muscle function in subjects in need thereof.

### SUMMARY

Provided is a method of administering a GLP-1 receptor agonist to a subject in need thereof, comprising administering an effective amount of the GLP-1 receptor agonist and further comprising administering simultaneously, separately, or sequentially in combination an effective amount of a 11β-hydroxysteroid dehydrogenase type 1 (HSD-1) inhibitor.

Also provided is a method of treating obesity in a subject in need thereof, comprising administering an effective amount of a GLP-1 receptor agonist and further comprising administering simultaneously, separately, or sequentially in combination an effective amount of a HSD-1 inhibitor.

Also provided is a method of inducing weight loss with maintenance of lean muscle mass in a subject in need of weight loss, comprising administering an effective amount of a GLP-1 receptor agonist and further comprising administering simultaneously, separately, or sequentially in combination an effective amount of a HSD-1 inhibitor so as to maintain lean muscle mass while inducing fat and weight loss in the subject.

Also provided is a method of inducing weight loss with maintenance of lean muscle mass in a subject in need of weight loss, comprising administering an effective amount of a GLP-1 receptor agonist and further comprising administering simultaneously, separately, or sequentially in combination an effective amount of a HSD-1 inhibitor so as to maintain lean muscle mass while inducing fat and weight loss in the subject.

Also provided is a method of increasing total weight loss caused by administration of a pre-determined amount of a GLP-1 receptor agonist to a subject in need thereof, the method comprising: co-administering to a subject in need thereof, simultaneously, separately, or sequentially, an effective amount of a HSD-1 inhibitor, to increase total weight loss in the subject relative to weight loss caused by administration of a pre-determined amount of a GLP-1 receptor agonist alone.

Also provided is a method of treating or preventing further muscle mass decrease caused by administration of a GLP-1 agonist to a subject in need thereof, the method comprising: adding an effective dose of an effective amount of a HSD-1 inhibitor to the GLP-1 agonist treatment regimen of a subject in need thereof, to treat or prevent further lean muscle mass decrease in the subject.

Also provided is a method of improving glycemic control in a subject in need thereof, comprising administering an effective amount of a GLP-1 receptor agonist and further comprising administering simultaneously, separately, or sequentially in combination an effective amount of a HSD-1 inhibitor.

Also provided is a method of reducing the risk of major adverse cardiovascular events in a subject with type 2 diabetes mellitus in need thereof, comprising administering an effective amount of a GLP-1 receptor agonist and further comprising administering simultaneously, separately, or sequentially in combination an effective amount of a HSD-1 inhibitor.

Also provided is a pharmaceutical composition comprising a GLP-1 receptor agonist, a HSD-1 inhibitor, and optionally a pharmaceutically acceptable excipient.

Also provided is a kit comprising a first container and a second container, the first container comprising a GLP-1 receptor agonist, and the second container comprising a HSD-1 inhibitor.

These and other aspects of the invention will be apparent upon reference to the following detailed description. To this end, various references are set forth herein which describe in more detail certain background information, procedures, compounds, or compositions, and are each hereby incorporated by reference in their entirety.

### BRIEF DESCRIPTION OF THE DRAWINGS

FIG. 1A and FIG. 1B depict results of the studies described in Example 1.

FIG. 2 depicts the schematic of the study described in Example 2.

FIG. 3 depicts the relationship of end-of-study (Study Day 26) body weight to treatment and baseline value in DIO mice treated with clofutriben and semaglutide. Confidence intervals of the least squares means were calculated using Dunnett's method.

FIG. 4A depicts end-of-study (Study Day 26) body lean mass absolute composition to treatment and baseline value in DIO mice treated with clofutriben and semaglutide. Confidence intervals of the least squares means were calculated using Dunnett's method.

FIG. 4B depicts end-of-study (Study Day 26) body fat mass absolute composition to treatment and baseline value in DIO mice treated with clofutriben and semaglutide. Confidence intervals of the least squares means were calculated using Dunnett's method.

FIG. 5A depicts end-of-study (Study Day 26) body lean mass percent composition to treatment and baseline value in DIO mice treated with clofutriben and semaglutide. Baseline body composition measurements obtained on Study Day-2 were normalized to body weight on Study Day 0. Confidence intervals of the least squares means were calculated using Dunnett's method.

FIG. 5B depicts end-of-study (Study Day 26) body fat mass percent composition to treatment and baseline value in DIO mice treated with clofutriben and semaglutide. Baseline body composition measurements obtained on Study Day-2 were normalized to body weight on Study Day 0. Confidence intervals of the least squares means were calculated using Dunnett's method.

FIG. 6A and FIG. 6B depict daily body weight & food consumption by treatment in DIO mice treated with clofutriben and semaglutide. Error bars are standard errors.

FIG. 7A and FIG. 7B depict post-mortem muscle weights by treatment in DIO mice treated with clofutriben and semaglutide. Diamonds represent means and 95% confidence intervals of the means.

FIG. 8A, FIG. 8B, FIG. 8C, and FIG. 8D depict post-mortem fat depot weights by treatment in DIO mice treated with clofutriben and semaglutide. Diamonds represent means and 95% confidence intervals of the means.

FIG. 9 depicts grip strength by treatment in DIO mice treated with clofutriben and semaglutide. Diamonds represent means and 95% confidence intervals of the means.

FIG. 10A, FIG. 10B, and FIG. 10C depict glycemic control parameters by treatment in DIO mice treated with clofutriben and semaglutide. Diamonds represent means and 95% confidence intervals of the means. Circles represent 95% confidence intervals of the means by Dunnett's method. Box plots represent medians, interquartile ranges, and range without outliers.

### DETAILED DESCRIPTION

### Methods

Provided are methods of administering a GLP-1 receptor agonist with a HSD-1 inhibitor to a subject in need thereof. In some examples, the method may include administering an effective amount of the GLP-1 receptor agonist and administering simultaneously, separately, or sequentially in combination an effective amount of a HSD-1 inhibitor.

Provided is a method of treating obesity in a subject in need thereof, comprising administering an effective amount of a GLP-1 receptor agonist and further comprising administering simultaneously, separately, or sequentially in combination an effective amount of a HSD-1 inhibitor.

Provided is a method of inducing weight loss with maintenance of lean muscle mass in a subject in need of weight loss, comprising administering an effective amount of a GLP-1 receptor agonist and further comprising administering simultaneously, separately, or sequentially in combination an effective amount of a HSD-1 inhibitor so as to maintain lean muscle mass while inducing fat and weight loss in the subject.

Provided is a method of increasing total weight loss caused by administration of a pre-determined amount of a GLP-1 receptor agonist to a subject in need thereof, the method comprising: co-administering to a subject in need thereof, simultaneously, separately, or sequentially, an effective amount of a HSD-1 inhibitor, to increase total weight loss in the subject relative to weight loss caused by administration of a pre-determined amount of a GLP-1 receptor agonist alone.

Provided is a method of treating or preventing further muscle mass decrease caused by administration of a GLP-1 agonist to a subject in need thereof, the method comprising: adding an effective dose of an effective amount of a HSD-1 inhibitor to the GLP-1 agonist treatment regimen of a subject in need thereof, to treat or prevent further lean muscle mass decrease in the subject.

Provided is a method of improving glycemic control in a subject in need thereof, comprising administering an effective amount of a GLP-1 receptor agonist and further comprising administering simultaneously, separately, or sequentially in combination an effective amount of a HSD-1 inhibitor.

Provided is a method of reducing the risk of major adverse cardiovascular events in a subject with type 2 diabetes mellitus in need thereof, comprising administering an effective amount of a GLP-1 receptor agonist and further comprising administering simultaneously, separately, or sequentially in combination an effective amount of a HSD-1 inhibitor.

In some embodiments, the GLP-1 receptor agonist is administered orally, intravenously, subcutaneously, intranasally, or intramuscularly.

In some embodiments, the GLP-1 receptor agonist is administered orally.

In some embodiments, the GLP-1 receptor agonist is administered parenterally.

In some embodiments, the GLP-1 receptor agonist is administered daily. In some embodiments, the GLP-1 receptor agonist is administered twice daily. In some embodiments, the GLP-1 receptor agonist is administered every other day. In some embodiments, the GLP-1 receptor agonist is administered weekly. In some embodiments, the GLP-1 receptor agonist is administered every other week. In some embodiments, the GLP-1 receptor agonist is administered monthly.

The desired dose of the GLP-1 receptor agonist may conveniently be presented in a single dose or as divided doses administered at appropriate intervals, for example, as two, three, four, or more sub-doses per day. The sub-dose itself may be further divided, e.g., into a number of discrete loosely spaced administrations. The daily dose can be divided, especially when relatively large amounts are administered as deemed appropriate, into several, for example two, three, or four-part administrations. If appropriate, depending on individual behavior, it may be necessary to deviate upward or downward from the daily dose indicated.

In some embodiments, the HSD-1 inhibitor is administered orally, intravenously, subcutaneously, intranasally, or intramuscularly.

In some embodiments, the HSD-1 inhibitor is administered orally.

In some embodiments, the HSD-1 inhibitor is administered parenterally.

In some embodiments, the HSD-1 inhibitor is administered daily. In some embodiments, the HSD-1 inhibitor is administered every other day. In some embodiments, the HSD-1 inhibitor is administered weekly. In some embodiments, the HSD-1 inhibitor is administered every other week. In some embodiments, the HSD-1 inhibitor is administered monthly.

The desired dose of the HSD-1 inhibitor may conveniently be presented in a single dose or as divided doses administered at appropriate intervals, for example, as two, three, four, or more sub-doses per day. The sub-dose itself may be further divided, e.g., into a number of discrete, loosely spaced administrations. The daily dose can be divided, especially when relatively large amounts are administered as deemed appropriate, into several, for example two, three, or four-part administrations. If appropriate, depending on individual behavior, it may be necessary to deviate upward or downward from the daily dose indicated.

In some embodiments, the HSD-1 inhibitor and the GLP-1 receptor agonist are administered simultaneously.

In some embodiments, the HSD-1 inhibitor and the GLP-1 receptor agonist are administered separately.

In some embodiments, the HSD-1 inhibitor and the GLP-1 receptor agonist are administered separately and sequentially.

In some examples, the GLP-1 receptor agonist may be administered at a different frequency than the HSD-1 inhibitor, such that at some times the GLP-1 receptor agonist and the HSD-1 inhibitor may be administered simultaneously or within minutes or hours of one another, while at other times the GLP-1 receptor agonist and the HSD-1 inhibitor may be administered on different days.

In some examples, the GLP-1 receptor agonist may be administered once weekly, and the HSD-1 inhibitor may be administered once daily. In other examples, the GLP-1 receptor agonist may be administered once daily, and the HSD-1 inhibitor may be administered once daily.

The GLP-1 receptor agonist and the HSD-1 inhibitor each may be administered with or without food.

In some embodiments, prior to administration of the HSD-1 inhibitor, the subject exhibits cortisol excess or one or more symptoms of glucocorticoid excess. The patient may have an elevated level of cortisol.

In some embodiments, prior to administration of the HSD-1 inhibitor, the subject exhibits cortisol non-suppression based on overnight dexamethasone suppression test (DST). In some embodiments, the overnight DST is chosen from a 1 mg overnight DST, a 8 mg overnight DST, a 2-day low dose DST, and a 2-day high dose DST. In some embodiments, a level greater than 5 microgram per deciliter (mcg/dl) is considered to indicate clinically relevant hypercortisolism. In some embodiments, a level greater than 1.8 microgram per deciliter (mcg/dl) is considered to indicate clinically relevant hypercortisolism. In some embodiments, a level greater than 1.4 microgram per deciliter (mcg/dl) or less is considered to indicate clinically relevant hypercortisolism. In some embodiments, a level greater than 1.2 microgram per deciliter (mcg/dl) is considered to indicate clinically relevant hypercortisolism.

In various examples, the patient may have a serum cortisol concentration of >about 1.2 μg/dL, >about 1.3 μg/dL, >about 1.4 μg/dL, >about 1.5 μg/dL, >about 1.6 μg/dL, >about 1.7 μg/dL, >about 1.8 μg/dL, >about 1.9 μg/dL, >about 2.0 μg/dL, >about 2.1 μg/dL, >about 2.2 μg/dL, >about 2.3 μg/dL, >about 2.4 μg/dL, >about 2.5 μg/dL, >about 2.6 μg/dL, >about 2.7 μg/dL, >about 2.8 μg/dL, >about 2.9 μg/dL, >about 3.0 μg/dL, >about 3.1 μg/dL, >about 3.2 μg/dL, >about 3.3 μg/dL, >about 3.4 μg/dL, >about 3.5 μg/dL, >about 3.6 μg/dL, >about 3.7 μg/dL, >about 3.8 μg/dL, >about 3.9 μg/dL, >about 4.0 μg/dL, >about 4.1 μg/dL, >about 4.2 μg/dL, >about 4.3 μg/dL, >about 4.4 μg/dL, >about 4.5 μg/dL, >about 4.6 μg/dL, >about 4.7 μg/dL, >about 4.8 μg/dL, or >about 5.0 μg/dL after an overnight DST.

In some embodiments, prior to administration of the HSD-1 inhibitor, the subject exhibits urinary free cortisol above a reference range.

In some embodiments, prior to administration of the HSD-1 inhibitor, the subject exhibits nighttime salivary cortisol above a reference range.

In some embodiments, prior to administration of the HSD-1 inhibitor, the subject exhibits an elevated urinary HSD-1 ratio. See, e.g., WO 2023/225507, which is incorporated herein by reference in its entirety.

In some embodiments, the subject has been diagnosed with endogenous Cushing's syndrome.

In some embodiments, the subject has been diagnosed with autonomous cortisol secretion.

In some embodiments, the subject is being chronically administered one or more glucocorticoid medicines. In some embodiments, the HSD-1 inhibitor or the GLP-1 receptor agonist is administered with the glucocorticoid medication at the same time (e.g., in a fixed-dose combination) or at a different time. In some embodiments, the HSD-1 inhibitor and the glucocorticoid medication are co-packaged for separate administration. In some embodiments, the HSD-1 inhibitor or the GLP-1 receptor agonist are administered and packaged separately from the glucocorticoid medication. In some embodiments, the glucocorticoid medication is selected from prednisolone, methylprednisolone, dexamethasone, hydrocortisone, budesonide, deflazacort, beclomethasone, ciclesonide, fluticasone, mometasone, triamcinolone, flunisolide, clobetasol, betamethasone, fluocinonide, flurandrenolide, clocortolone, halobetasol, desoximetasone, desonide, halcinonide, prednicarbate, diflorasone, amcinonide, alclometasone, difluprednate, loteprednol, fluorometholone, rimexolone, and medrysone, or a pharmaceutically acceptable salt or ester derivative thereof, or combinations thereof.

In some embodiments, the subject has an adrenal tumor.

In some embodiments, prior to administration of the HSD-1 inhibitor, the subject is at risk of experiencing muscle loss associated with GLP-1 receptor agonist administration.

In some embodiments, the subject has a sedentary lifestyle, osteopenia or osteoporosis, poor nutrition, or is at an advanced age.

In some embodiments, the subject is unable to engage in strength and resistance training.

In some embodiments, the subject has a muscle-wasting condition such as chronic kidney disease.

In some embodiments, the subject is on a calorie restriction diet or an exercise regimen.

In some embodiments, the administering of the HSD-1 inhibitor preserves muscle mass, or stimulates an increase in muscle mass in the subject relative to a baseline level.

In some embodiments, the subject exhibits after the administration of the HSD-1 inhibitor and the GLP-1 receptor agonist one or more effects chosen from: loss of fat but not lean muscle; increased lean mass percentage; increased lean/fat mass ratio; and reduced or normal fed glucose level, relative to a baseline level immediately before administration.

In some embodiments, the subject is overweight or obese.

In some embodiments, the subject has a disease or condition associated with weight gain.

In some embodiments, the disease or condition associated with weight gain is selected from obesity, obesity-linked gallbladder disease, obesity-induced sleep apnea, diabetes, excessive appetite, fatty liver disease, non-alcoholic fatty liver disease (NASH), dyslipidemia, metabolic syndrome, insufficient satiety, hyperinsulinemia, and nighttime hypoglycemia.

In some embodiments, the subject has a metabolic disorder.

In some embodiments, the subject has diabetic obesity.

In some embodiments, the subject has type 1 diabetes, type 2 diabetes, or gestational diabetes.

In some embodiments, the subject has one or more of hypertension, dyslipidemia, obstructive sleep apnea, and cardiovascular disease.

In some embodiments, the treatment decreases the patient's hemoglobin A1c (HbA1c) levels compared to the patient's HbA1c levels before the GLP-1 receptor agonist and HSD-1 inhibitor are initially administered to the patient.

### HSD-1 Inhibitors

A number of HSD-1 inhibitors are known and available in the art, including, but not limited to, SPI-62, ABT-384, MK-0736, MK-0916, BMS-823778, UE-2343, AMG-221, KR-67607, BI-187004, SAR-184481, Compound A, Compound B, Compound C, Compound D, BMS-823778, or a pharmaceutically acceptable salt thereof. As would be understood by one of skill in the art, any HSD-1 inhibitor described herein or known or available in the art may be used as described herein and is encompassed within the scope of the present disclosure.

The structures of some HSD-1 inhibitors described herein are set forth below. All structures and IUPAC names were generated using ChemDraw 20.0.

As used herein, “SPI-62” and “clofutriben” refers to 4-(5-(2-(4-chloro-2,6-difluorophenoxy) propan-2-yl)-4-methyl-4H-1,2,4-triazol-3-yl)-3-fluorobenzamide. SPI-62 is also referred to as clofutriben.

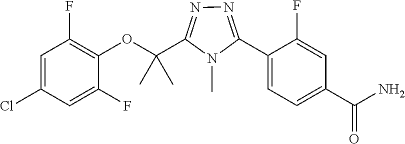 [SMILES: Cn1c(-c2ccc(C(N)=O)cc2F)nnc1C(C)(C)Oc1c(F)cc(Cl)cc1F]

As used herein, “BMS-823778” refers to 2-(3-(1-(4-chlorophenyl)cyclopropyl)-[1,2,4]triazolo[4,3-a]pyridin-8-yl) propan-2-ol.

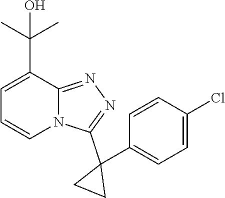 [SMILES: CC(C)(O)c1cccn2c(C3(c4ccc(Cl)cc4)CC3)nnc12]

As used herein, “ABT-384” refers to (1s,3R,4r,5S,7s)-4-(2-methyl-2-(4-(5-(trifluoromethyl)pyridin-2-yl) piperazin-1-yl) propanamido) adamantane-1-carboxamide.

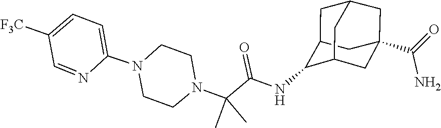 [SMILES: CC(C)(C(=O)N[C@H]1C2CC3CC1C[C@@](C(N)=O)(C3)C2)N1CCN(c2ccc(C(F)(F)F)cn2)CC1]

As used herein, “UE-2343” refers to (5-(1H-pyrazol-4-yl)thiophen-3-yl)((1R,3r,5S)-3-hydroxy-3-(pyrimidin-2-yl)-8-azabicyclo[3.2.1]octan-8-yl) methanone.

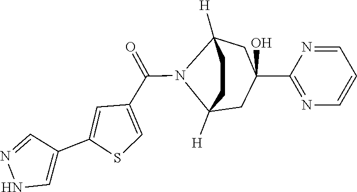

As used herein, “MK-0736” refers to 3-(4-(3-(ethylsulfonyl) propyl) bicyclo[2.2.2]octan-1-yl)-4-methyl-5-(2-(trifluoromethyl)phenyl)-4H-1,2,4-triazole.

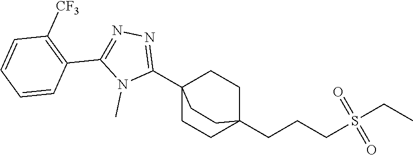 [SMILES: CCS(=O)(=O)CCCC12CCC(c3nnc(-c4ccccc4C(F)(F)F)n3C)(CC1)CC2]

As used herein, “AMG-221” refers to (S,E)-2-(((1S,2S,4R)-bicyclo[2.2.1]heptan-2-yl)imino)-5-isopropyl-5-methylthiazolidin-4-one

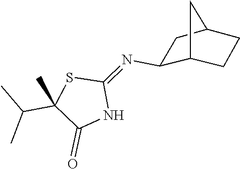 [SMILES: CC(C)[C@]1(C)S/C(=N/C2CC3CCC2C3)NC1=O]

As used herein, “MK-0916” refers to 3-((1s,3s)-1-(4-chlorophenyl)-3-fluorocyclobutyl)-4,5-dicyclopropyl-4H-1,2,4-triazole.

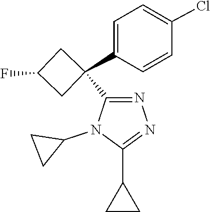 [SMILES: F[C@H]1C[C@@](c2ccc(Cl)cc2)(c2nnc(C3CC3)n2C2CC2)C1]

As used herein, “KR-67607” refers to (1s,3R,4s,5S,7s)-4-(2-(6-(2,6-dichloro-4-(trifluoromethyl)phenyl)-4-methyl-1,1-dioxido-1,2,6-thiadiazinan-2-yl) acetamido) adamantane-1-carboxamide.

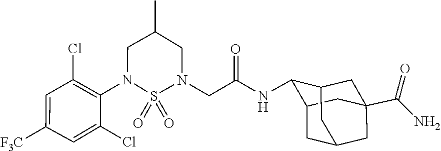 [SMILES: CC1CN(CC(=O)NC2C3CC4CC2CC(C(N)=O)(C4)C3)S(=O)(=O)N(c2c(Cl)cc(C(F)(F)F)cc2Cl)C1]

As used herein, “BI-187004” refers to (4aR,9aS)-1-(1H-benzo[d]imidazole-6-carbonyl)-2,3,4,4a,9,9a-hexahydro-1H-indeno[2,1-b]pyridine-6-carbonitrile.

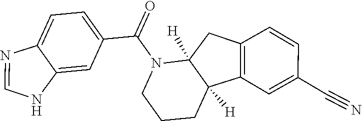 [SMILES: N#Cc1ccc2c(c1)[C@H]1CCCN(C(=O)c3ccc4nc[nH]c4c3)[C@H]1C2]

As used herein, “AZD4017” refers to(S)-2-(1-(5-(cyclohexylcarbamoyl)-6-(propylthio)pyridin-2-yl) piperidin-3-yl) acetic acid.

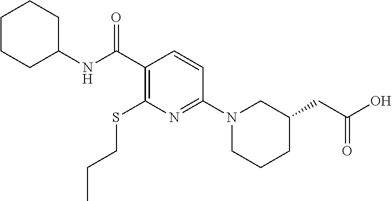 [SMILES: CCCSc1nc(N2CCC[C@@H](CC(=O)O)C2)ccc1C(=O)NC1CCCCC1]

As used herein, “AZD8329” refers to 4-(4-(((1r,3r,5r,7r)-adamantan-2-yl) carbamoyl)-5-(tert-butyl)-1H-pyrazol-1-yl)benzoic acid.

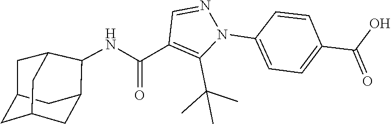

As used herein, “BI-135585” refers to(S)-6-(2-hydroxy-2-methylpropyl)-3-((S)-1-(4-(1-methyl-2-oxo-1,2-dihydropyridin-4-yl)phenyl)ethyl)-6-phenyl-1,3-oxazinan-2-one.

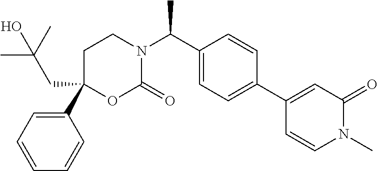 [SMILES: C[C@@H](c1ccc(-c2ccn(C)c(=O)c2)cc1)N1CC[C@](CC(C)(C)O)(c2ccccc2)OC1=O]

As used herein, “SAR-184841” refers to 4-(5-(4-(tert-butyl) piperazin-1-yl)pyridin-2-yl)-N-((1R,3S,5s,7s)-5-carbamoyladamantan-2-yl)-3,4-dihydroquinoxaline-1(2H)-carboxamide

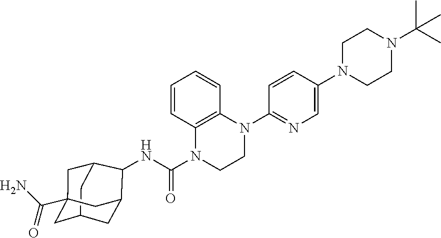 [SMILES: CC(C)(C)N1CCN(c2ccc(N3CCN(C(=O)NC4C5CC6CC4CC(C(N)=O)(C6)C5)c4ccccc43)nc2)CC1]

As used herein, “Compound A” refers to (1s,3R,5S,7s)-4-(2-(4-methoxyphenoxy)-2-methylpropanamido) adamantane-1-carboxamide

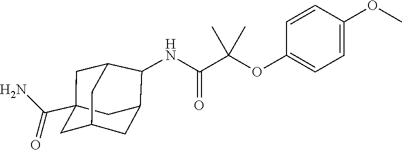 [SMILES: COc1ccc(OC(C)(C)C(=O)NC2C3CC4CC2CC(C(N)=O)(C4)C3)cc1]

As used herein, “Compound B” refers to 3,4-dihydroquinolin-1(2H)-yl[4-(1H-imidazol-5-yl) piperidin-1-yl]methanone.

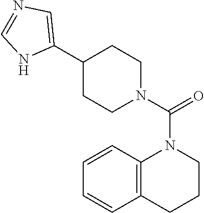 [SMILES: O=C(N1CCC(c2cnc[nH]2)CC1)N1CCCc2ccccc21]

As used herein, “Compound C” refers to (5R)-2-[(2-fluorophenyl)amino]-5-(1-methylethyl)-1,3-thiazol-4(5H)-one.

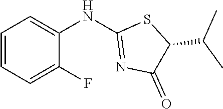 [SMILES: CC(C)[C@H]1SC(Nc2ccccc2F)=NC1=O]

As used herein, “Compound D” refers to 3-(2-fluoroethyl)-4-((4-[(2S)-1,1,1-trifluoro-2-hydroxypropan-2-yl]phenyl)sulfonyl)benzonitrile.

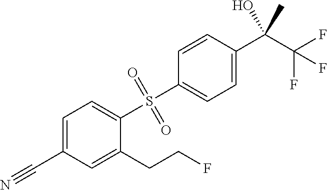 [SMILES: C[C@](O)(c1ccc(S(=O)(=O)c2ccc(C#N)cc2CCF)cc1)C(F)(F)F]

In some embodiments, the HSD-1 inhibitor is a pseudo-irreversible inhibitor. In some embodiments, the pseudo-irreversible HSD-1 inhibitor does not show tachyphylaxis for human adipose HSD-1 inhibition. In some embodiments, the pseudo-irreversible HSD-1 inhibitor is characterized by human pharmacokinetics consistent with target-mediated drug disposition. In some embodiments, plasma exposures of the pseudo-irreversible HSD-1 inhibitor are less than dose-proportional after low single doses and dose-proportional after multiple low doses. In some embodiments, the pseudo-irreversible HSD-1 inhibitor is characterized by human pharmacodynamics consistent with target-mediated drug-disposition. In some embodiments, the pharmacodynamic half-life of the HSD-1 inhibitor for hepatic HSD-1 inhibition is extended compared to its pharmacokinetic half-life. In some embodiments, the pseudo-irreversible HSD-1 inhibitor is characterized by a pharmacodynamic half-life for human hepatic HSD-1 inhibition of at least 1 week. In some embodiments, the pharmacodynamic half-life for human hepatic HSD-1 inhibition is at least 2 weeks. In some embodiments, the pharmacodynamic half-life for human hepatic HSD-1 inhibition is at least 4 weeks. In some embodiments, the pseudo-irreversible HSD-1 inhibitor is characterized by fast-on, slow-off in vitro binding kinetics to human HSD-1. In some embodiments, the residence time of the HSD-1 inhibitor is at least about 500 seconds. In some embodiments, the pseudo-irreversible HSD-1 inhibitor is characterized by apparent fast-on, slow-off in vivo binding kinetics to human HSD-1. In some embodiments, the model-estimated k_{off} is less than 0.3 h⁻¹. In some embodiments, the model-estimated k_{off} is less than 1.0 h⁻¹. In some embodiments, the model-estimated k_{off} is less than 3.0 h⁻¹. In some embodiments, the pseudo-irreversible HSD-1 inhibitor is characterized by having a greater potency in vivo than in vitro. In some embodiments, the model-estimated K_{d} of the HSD-1 inhibitor is lower than that of the HSD-1 inhibitor Kᵢ. In some embodiments, the model-estimated K_{d} is at least 50-fold lower than Kᵢ. In some embodiments, the model-estimated K_{d} is at least 100-fold lower than that Kᵢ. In some embodiments, the model-estimated K_{d} is at least 200-fold lower than Kᵢ. In some embodiments, the model-estimated adipose IC₅₀ of the HSD-1 inhibitor is lower than that of the HSD-1 inhibitor measured in vitro. In some embodiments, the model-estimated adipose IC₅₀ is at least 100-fold lower than that measured in vitro. In some embodiments, the model-estimated adipose IC₅₀ is at least 300-fold lower than that measured in vitro. In some embodiments, the model-estimated adipose IC₅₀ is at least 700-fold lower than that measured in vitro. In some embodiments, the pseudo-irreversible HSD-1 inhibitor forms hydrogen bonds to the pyrophosphate of NADPH in the human HSD-1 active site.

In some embodiments, the HSD-1 inhibitor is clofutriben.

In some embodiments, the HSD-1 inhibitor is administered in a single daily dose of from about 1 mg to about 40 mg, including 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, or the like. In some embodiments, a sub-milligram dose may be any dose about equal to, or less than, 1 mg, such as 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, or 1 mg.

In some embodiments, the HSD-1 inhibitor may be administered in multiple doses for a specified period of time. Multiple doses may refer to multiple doses in a single day, or, alternatively, may refer to single doses per day for a period of time deemed appropriate by a clinician or practitioner.

In some embodiments, a dose range for clofutriben is from about 0.2 to about 40 mg, about 0.2 mg to about 20 mg, or about 0.2 mg to about 10 mg once daily. In some embodiments, the dose of clofutriben is about 0.6 mg, about 2 mg, about 6 mg, or about 12 mg once daily. In other examples, the daily dose of clofutriben may be about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3.0 mg, about 3.1 mg, about 3.2 mg, about 3.3 mg, about 3.4 mg, about 3.5 mg, about 3.6 mg, about 3.7 mg, about 3.8 mg, about 3.9 mg, about 4.0 mg, about 4.1 mg, about 4.2 mg, about 4.3 mg, about 4.4 mg, about 4.5 mg, about 4.6 mg, about 4.7 mg, about 4.8 mg, about 4.9 mg, about 5.0 mg, about 5.1 mg, about 5.2 mg, about 5.3 mg, about 5.4 mg, about 5.5 mg, about 5.6 mg, about 5.7 mg, about 5.8 mg, about 5.9 mg, about 6.0 mg, about 6.1 mg, about 6.2 mg, about 6.3 mg, about 6.4 mg, about 6.5 mg, about 6.6 mg, about 6.7 mg, about 6.8 mg, about 6.9 mg, about 7.0 mg, about 7.1 mg, about 7.2 mg, about 7.3 mg, about 7.4 mg, about 7.5 mg, about 7.6 mg, about 7.7 mg, about 7.8 mg, about 7.9 mg, about 8.0 mg, about 8.1 mg, about 8.2 mg, about 8.3 mg, about 8.4 mg, about 8.5 mg, about 8.6 mg, about 8.7 mg, about 8.8 mg, about 8.9 mg, about 9.0 mg, about 9.1 mg, about 9.2 mg, about 9.3 mg, about 9.4 mg, about 9.5 mg, about 9.6 mg, about 9.7 mg, about 9.8 mg, about 9.9 mg, about 10.0 mg, about 10.1 mg, about 10.2 mg, about 10.3 mg, about 10.4 mg, about 10.5 mg, about 10.6 mg, about 10.7 mg, about 10.8 mg, about 10.9 mg, about 11.0 mg, about 11.1 mg, about 11.2 mg, about 11.3 mg, about 11.4 mg, about 11.5 mg, about 11.6 mg, about 11.7 mg, about 11.8 mg, about 11.9 mg, about 12.0 mg, about 12.1 mg, about 12.2 mg, about 12.3 mg, about 12.4 mg, about 12.5 mg, about 12.6 mg, about 12.7 mg, about 12.8 mg, about 12.9 mg, or about 13.0 mg.

In some embodiments, a period of time for administration of the HSD-1 inhibitor may be any period of time, such as including, but not limited to, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, about 4 weeks, about 6 weeks, about 8 weeks, about 10 weeks, about 12 weeks, more than 12 weeks, or the like. A period of time in accordance with the present disclosure may be, any period of time, for example, a period of 1-14 days, or 7-14 days, or 7-10 days, or 10-14 days. In other embodiments, a period of time for administration of the HSD-1 inhibitor may be from 7-30 days, 7-21 days, 1 week to 4 weeks, 4 weeks to 8 weeks, 4 weeks to 12 weeks, or the like. In some embodiments, a period of time for administration of an HSD-1 inhibitor may be 1 day, 6 days, or 12 days. In some embodiments, the HSD-1 inhibitor may be administered to a patient on a continual basis to control hypercortisolism. In some embodiments, administration of a dosage of the HSD-1 inhibitor may be for 1 month, or 2 months, or 3 months, or 4 months, or 5 months, or 6 months, or 7 months, or 8 months, or 9 months, or 10 months, or 11 months, or 12 months, or 15 months, or 18 months, or 24 months, or 36 months, or 48 months, or longer. In some embodiments, the HSD-1 inhibitor may be administered intravenously in a single high dose, for example to control sepsis or to prevent the development of side effects of steroid use, e.g., in diabetic patients.

In some embodiments, a HSD-1 inhibitor as described herein may be administered by any route deemed appropriate by a practitioner or clinician. Suitable routes of administration include, but are not limited to, oral, intravenous, intramuscular, subcutaneous, inhalation, intranasal, ocular, topical.

### GLP-1 Receptor Agonist

In some embodiments, the GLP-1 receptor agonist is a dual-acting GLP-1 agonist, and glucose-dependent insulinotropic polypeptide (GIP) receptor agonist or glucagon receptor agonist.

In some embodiments, the GLP-1 agonist is a triple-acting GLP-1 receptor agonist, GIP receptor agonist, and glucagon receptor agonist.

In some embodiments, the GLP-1 agonist is selected from albiglutide, dulaglutide, exenatide, liraglutide, lixisenatide, semaglutide, and tirzepatide.

In some embodiments, the GLP-1 agonist is albiglutide. In some embodiments, the GLP-1 agonist is albiglutide administered 30 mg subcutaneously once weekly. In some embodiments, the GLP-1 agonist is albiglutide administered 50 mg subcutaneously once weekly.

In some embodiments, the GLP-1 agonist is dulaglutide. In some embodiments, the GLP-1 agonist is dulaglutide administered to an adult at 0.75 mg injected subcutaneously once weekly. In some embodiments, the dosage may be increased to 1.5 mg once weekly for additional glycemic control. In some embodiments, the dosage is increased in 1.5 mg increments after at least 4 weeks on the current dosage to a maximum recommended dosage of 4.5 mg injected subcutaneously once weekly. In some embodiments, the GLP-1 agonist is dulaglutide administered to a pediatric subject at 0.75 mg injected subcutaneously once weekly. In some embodiments, the dosage is increased to a maximum recommended dosage of 1.5 mg injected subcutaneously once weekly.

In some embodiments, the GLP-1 agonist is exenatide. In some embodiments, the GLP-1 agonist is exenatide administered 5 mcg per dose twice daily and optionally increased to 10 mcg twice daily after 1 month based on clinical response.

In some embodiments, the GLP-1 agonist is liraglutide. In some embodiments, the GLP-1 agonist is liraglutide administered at 0.6 mg per day for one week then increased to 1.2 mg. The dose can be increased to 1.8 mg for additional glycemic control

In some embodiments, the GLP-1 agonist is lixisenatide. In some embodiments, the GLP-1 agonist is lixisenatide administered at 10 mcg once daily for 14 days and then at an increased dosage of 20 mcg once daily.

In some embodiments, the GLP-1 agonist is semaglutide. In some embodiments, the GLP-1 agonist is semaglutide administered at 0.25 mg once weekly. After 4 weeks, the dose is increased to 0.5 mg once weekly. In some embodiments, the dose is increased to 1 mg once weekly.

In some embodiments, the GLP-1 agonist is tirzepatide. In some embodiments, the GLP-1 agonist is tirzepatide administered 2.5 mg injected subcutaneously once weekly. In some embodiments, the dosage is increased in 2.5 mg increments after at least 4 weeks on the current dose to a maximum dosage of 15 mg subcutaneously once weekly

In some embodiments, the GLP-1 agonist is selected from albenatide, avexitide, cafraglutide, cotadutide, danuglipron, dapiglutide, diabegone, dulaglutide, ecnoglutide, efpeglenatide, efinopegdutide, efocipegtrutide, froniglutide, CT-868, efocipegtrutide, LY-3502970, maridebart, mazdutide, NLY-001, orforglipron, pegapamodutide, pemvidutide, retatrutide (LY-3437943), survodutide, vurolenatide, dapagliflozin+semaglutide, (cagrilintide+semaglutide), (LAI-287+semaglutide), (semaglutide+GIP analogue), 4P-004, AMG-133, AP-026, AZD-9550, BGM-0504, BMS-686117, Zn/BMS-686117 adduct, CT-388, CT-868, CT-996, DD-01, DR-10624, DR-10627, ECC-5004, E-2HSA, GL-0034, GLP-06, GMA-105, GMA-106, GMA-102, GSBR-1290, GXG-6, GZR-18, HEC-88473, HR-17031, HRS-7535, HRS-9531, HS-20004, HS-20094, HB-1085, HDM-1002, HL-08, HZ-010, JY-09, KN-056, LY-3493269, MBX-1416, MDR-001, MWN-101, NLY-001, NN-9490, NNC0519-0130, NN-6177, NN-9847, NN-9904, PF-06954522, PF-07081532, PF-06882961, PB-1023, PB-119, PB-718, RGT-075, SAL-015, SAL-0112, SCO-094, TERN-601, TTP-273, Uni-E4, VK-2735, YH-25724, XW-004, XW-014, YH-25724, YN-012, YN-015, ZT-002, and pharmaceutically acceptable salts thereof.

In some embodiments, the GLP-1 receptor agonist is selected from (dorzagliatin+GLP-1), (exenatide+insulin aspart), ACT-1003, Adogel Sema, AER-601, AGM-212, BEBT-808, BZ-043B, C-2816, DAJC-1, DD-02, DR-10625, DR-10628, DS-004, DS-005, DS-006, DS-012, E-6, efpeglenatide+HM-12470, exenatide 2, exenatide LA, exenatide SR, Extendin-Fc, G-49, GB-7001, Gene Encoding GLP-1, GLP-1 Incretin Triagonist, GLP-1 Oral Preparation, GLP-1R Antagonist for Hypoglycemia, glucagon, Glucagon-Like Peptide-1+insulin human, GPCR-targeted Project 012, GPCR-targeted Project 013, GT-01123, HM-15275, HPG-5119, HSP-001, HSP-004, HSP-005, HSP012-C, Hydrogel Exenatide, 120-105S, 120-110, KP-405, LA-EX, liraglutide biobetter, liraglutide LA, MK-1462, MLX-7000, MWN-105, MWN-109, NLY-12, NPM-115, OGB-21502, OXM, P-11, PB-2301, PB-2309, RGT-028, RGT-274, RPC-8844, RT-104, SHX-022, SL-209, synthetic peptides to agonize GLP-1R and CCKBR for diabetes, TB-013, TB-222023, TB-592, TE-8105, THDBH-111, UDS-003, VTCG-15, XL-110, XL-310, XW-003+XW-015, XW-003+XW-017, Y-002, YGX-1, ZT-003, ZT-006, ZT-007, DA-1726, HDM-1005, (insulin degludec+liraglutide), DB-081, GW-002, HZCX-012, ID-110521156, THDB-0211, THDBH-110, THDBH-120, THDBH-121, UBT-251, ATBB-22, BEM-012, CIN-209, CIN-210, DD-03, exenatide+ND-017, exenatide+Synthetic Peptide 2, glucagon, Insulin-GLP1, MD-02, OGB-21501, P-01, PAT-201, PF-1807, PT-3, and pharmaceutically acceptable salts thereof.

In some embodiments, the GLP-1 receptor agonist is selected from albiglutide, beinaglutide, dulaglutide, exenatide, liraglutide, lixisenatide, semaglutide, tirzepatide, orforglipron, mazdutide, danuglipron, lotiglipron, pemvidutide, mazdutide, CagriSema (combination of cagrilintide and semaglutide), retatrutide, PEG-loxenatide, and efpeglenatide.

### Compositions

Also provided is a pharmaceutical composition comprising a GLP-1 receptor agonist, a HSD-1 inhibitor, and optionally a pharmaceutically acceptable excipient. In some embodiments, the pharmaceutical composition further comprises one or more pharmaceutically acceptable excipients. Conventional excipients, such as binding agents, fillers, acceptable wetting agents, tableting lubricants, and disintegrants can be used in tablets and capsules for oral administration.

Compositions for oral administration can be in the form of solutions, emulsions, aqueous or oily suspensions, and syrups. Alternatively, the compositions for oral administration can be in the form of dry powder that can be reconstituted with water or another suitable liquid vehicle before use. Additional additives such as suspending or emulsifying agents, non-aqueous vehicles (including edible oils), preservatives, and flavorings and colorants can be added.

Compositions for intravenous or intramuscular administration can be prepared by dissolving the compounds in a suitable liquid vehicle and filter sterilizing the solution before filling and sealing an appropriate vial or ampoule. These are just a few examples of the many appropriate methods well known in the art for preparing compositions for oral, intravenous or intramuscular administration.

Also provided is a kit comprising a first container and a second container, the first container comprising a GLP-1 receptor agonist, and the second container comprising a HSD-1 inhibitor.

### Definitions

The definitions and methods provided define the present disclosure and guide those of ordinary skill in the art in the practice of the present disclosure. Unless otherwise noted, terms are to be understood according to conventional usage by those of ordinary skill in the relevant art.

As used herein, a “receptor agonist” refers to a compound that binds to a receptor and elicits a response typical of the natural ligand. As used herein, a “full agonist” may refers to a receptor agonist that elicits a response of the same magnitude as the natural ligand. Thus, for example, a “GLP-1 receptor agonist” or “GLP-1 agonist” refers to a compound which is capable of binding to the GLP-1 receptor and capable of activating it and a “full” GLP-1 agonist refers to a GLP-1 agonist which is capable of eliciting a magnitude of GLP-1 receptor response that is similar to native GLP-1.

As used herein, a “HSD-1 inhibitor” refers to a compound, such as a small molecule compound that bind to, interacts with, elicits a conformational change, or otherwise inhibits or abolishes the activity of HSD-1.

As used herein, “simultaneously” refers to administration of the HSD-1 inhibitor and the GLP-1 receptor agonist by the same route and at the same time or at substantially the same time.

As used herein, “separately” refers to administration of the HSD-1 inhibitor and the GLP-1 receptor agonist at the same time or at substantially the same time by different routes.

As used herein, “sequentially” refers to administration of the HSD-1 inhibitor and the GLP-1 receptor agonist at different times, the administration route being identical or different. More particularly, sequential administration method includes but is not limited to administration of to one of the active ingredients in its entirety before administration of the other or others commences. It is thus possible to administer one of the active ingredients over several months before administering the other active ingredient or ingredients. There is no simultaneous treatment in this case. An alternate administration of each active ingredient over several weeks can also be envisaged.

As used herein, an “effective amount” of a compound refers to an amount that is sufficient to generate a desired response, such as reduce or eliminate a sign or symptom of a condition or disease. In some examples, an “effective amount” is one that treats one or more symptoms. In one example, an effective amount is a therapeutically effective amount.

As used herein, “subject” or “patient” refers to a human. In some examples, the human patient may be an adult or pediatric patient (i.e., 18 years or younger). An adult patient is ≥18 years. The patient may have any of the conditions described herein. In some examples, a patient or subject in accordance with the present disclosure has cortisol excess or has one or more symptoms of glucocorticoid excess. Unless otherwise noted, the terms “patient” or “subject” are used herein interchangeably.

As used herein, a “reference sample” or “reference patient” is a sample or patient to whom a HSD-1 inhibitor was not administered as described herein, or a healthy patient.

As used herein, the term “treating” or “alleviating” includes the administration of compounds or agents to a patient for alleviating or reversing the symptoms, or arresting or inhibiting further development, of a disease, condition, or disorder. Subjects in need of treatment include those already suffering from the disease or disorder or experiencing symptoms of a disease, condition, or disorder, e.g., patients having elevated levels of glucocorticoids or an increase of the urinary ratio of cortisol metabolites (tetrahydrocortisol+allotetrahydrocortisol)/tetrahydrocortisone.

As used herein, the term “reference range” is meant to identify the reference or normal range as established by a laboratory and can vary per laboratory. Reference ranges may be specified by sex or age.

In some embodiments, the upper limit of the reference range for urinary free cortisol is between 16 and 100 micrograms per 24 hours. In some embodiments, the upper limit of the reference range for urinary free cortisol is 45 micrograms per 24 hours. In some embodiments, the upper limit of the reference range for urinary free cortisol is 60 micrograms per 24 hours.

In some embodiments, the upper limit of the reference range for late night salivary cortisol is between 0.97 and 1.45 micrograms per liter. In some embodiments, upper limit of the reference range for late night salivary cortisol is 1.00 micrograms per liter.

As used herein, “hypercortisolism” refers to a condition or physiological state in an individual in which the levels of cortisol is elevated relative to a reference patient or sample, or relative to a patient not experiencing hypercortisolism.

Numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes a measure of variability for the device or method being employed to determine the value. The term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. The numerical parameters set forth in the written description and attached claims are approximations that can vary depending upon the desired properties sought to be obtained by a particular embodiment. The endpoint may also be based on the variability allowed by an appropriate regulatory body, such as the FDA, USP, etc. The numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the variability found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

The terms “a,” and “an,” and “the,” and similar references used in the context of describing a particular embodiment (especially in the context of certain of the following claims) can be construed to cover both the singular and the plural, unless specifically noted otherwise. The term “or” as used herein, including the claims, is used inclusively unless explicitly indicated to refer to alternatives only or the alternatives are mutually exclusive.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1. Dose Range Finding for Clofutriben in Diet-Induced Obese Mouse

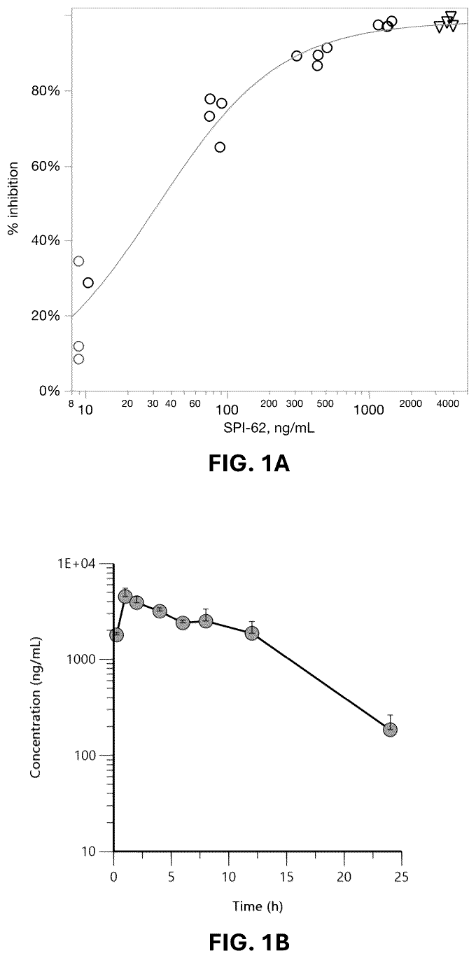

Male C57/BL6 mice received a high fat diet starting at 6 weeks of age. At 21 weeks of age, those diet-induced obese (DIO) mice were randomized to receive a single dose of clofutriben 0.03, 0.1, 0.3, 1, 3, 10, or 30 mg/kg or vehicle. At 90 minutes after the clofutriben dose, the mice received a single dose of prednisone 3 mg/kg. At 120 minutes after the clofutriben dose, plasma samples for measurement of prednisone, prednisolone, and clofutriben were obtained. The ratio prednisolone/prednisone was calculated. HSD-1 inhibition (%) was calculated as (Rv−Rc)/Rv, where Rv is the mean ratio for vehicle-treated mice and Rc is the ratio for a clofutriben-treated mouse. A Michaelis-Menten model was used to estimate the clofutriben concentration associated with 90% HSD-1 inhibition as 341 ng/mL (95% confidence interval 210-471 ng/ml). The results are provided in Table 1 and in FIG. 1A.

TABLE 1
Clofutriben concentration associated
with HSD-1 inhibition Predicted

| Specified % | Predicted | Std | Lower | Upper |
|---|---|---|---|---|
| inhibition | SPI-62, ng/mL | Error | 95% | 95% |
| 0.5000000 | 32.8860 | 3.80842 | 25.4216 | 40.3503 |
| 0.8000000 | 138.3717 | 16.10627 | 106.8040 | 169.9395 |
| 0.9000000 | 340.8203 | 66.65557 | 210.1778 | 471.4628 |

Male C57/BL6 mice received a high fat diet starting at 6 weeks of age. At 21 weeks of age, those diet-induced obese (DIO) mice received a single dose of clofutriben 10 mg/kg. Plasma samples for measurement of clofutriben were obtained at 15 minutes and 1, 2, 4, 6, 8, 12, and 24 hours after the clofutriben dose. Visual inspection of the clofutriben concentration-time plot, as seen in FIG. 1B, indicated that HSD-1 inhibition associated with clofutriben 10 mg/kg would be >90% for ~20 hours after a single dose and for all or nearly all of a 24-hour dose interval at steady-state.

### Example 2. Effects of Clofutriben and Semaglutide in Diet-Induced Obese Mouse

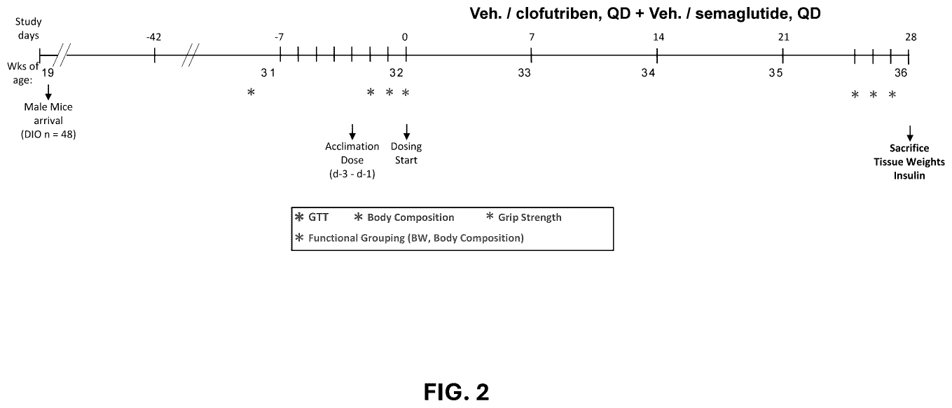

A study for administering clofutriben and semaglutide as shown in the schematic in FIG. 2 was performed.

Male C57BL6 mice received a high fat diet starting at 6 weeks of age. At 32 weeks of age on Study Day 0, those diet-induced obese (DIO) mice were randomized to treatment with clofutriben 10 mg/kg or vehicle once daily, and semaglutide 0.04 mg/kg once daily, for 4 weeks. Baseline assessments were intraperitoneal glucose tolerance test (ipGTT) at Study Day-15, whole body muscle and fat composition by magnetic resonance imaging at Study Day-2, and grip strength at Study Day-1. Body weight and food consumption were recorded daily throughout the study. Final in-life assessments were ipGTT at Study Day 25, body composition at Study Day 26, and grip strength at Study Day 27. Post-mortem assessments on Study Day 28 were fasting glucose and insulin measurements for calculation of Homeostatic Model Assessment of Insulin Resistance (HOMA-IR) and weights of selected skeletal muscles and adipose depots.

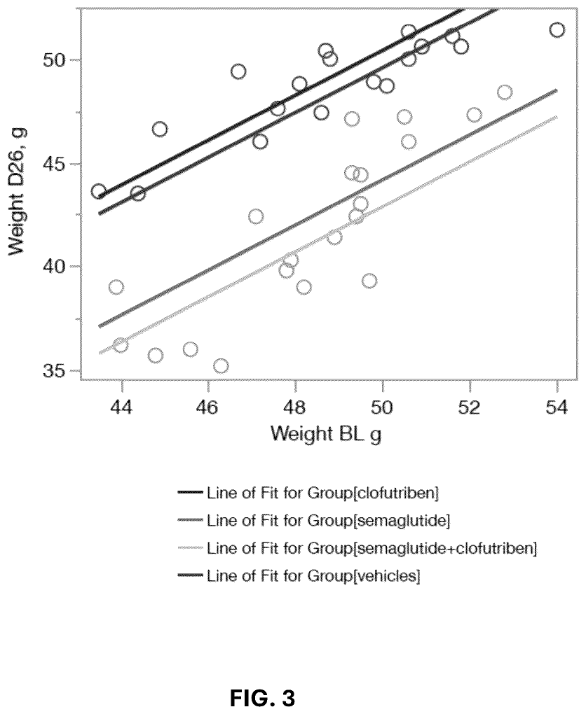

FIG. 3 and Tables 2 and 3 depict the relationship of end-of-study (Study Day 26) body weight to treatment and baseline value in DIO mice treated with clofutriben and semaglutide.

TABLE 2

| | Least | | | | | |
|---|---|---|---|---|---|---|
| Level | Sq Mean | Std Error | 35 | 40 | 45 | 50 |

| | | | |
|---|---|---|---|
| clofutriben | 48.8413 | 0.6720 | [symbol] |

| semaglutide | 42.5863 | 0.6358 | [symbol] | |
|---|---|---|---|---|
| semaglutide + clofutriben | 41.2986 | 0.6365 | [symbol] | |
| vehicles | 48.0088 | 0.6699 | | [symbol] |

TABLE 3

| | | | Adjusted | Adjusted | |
|---|---|---|---|---|---|
| Group | −Group | Difference | Lower 95% | Upper 95% | t Ratio |
| clofutriben | semaglutide | 6.25501 | 3.97166 | 8.538366 | 6.75 |
| semaglutide + clofutriben | semaglutide | −1.28768 | −3.50441 | 0.929040 | −1.43 |
| vehicles | semaglutide | 5.42256 | 3.14506 | 7.700050 | 5.87 |

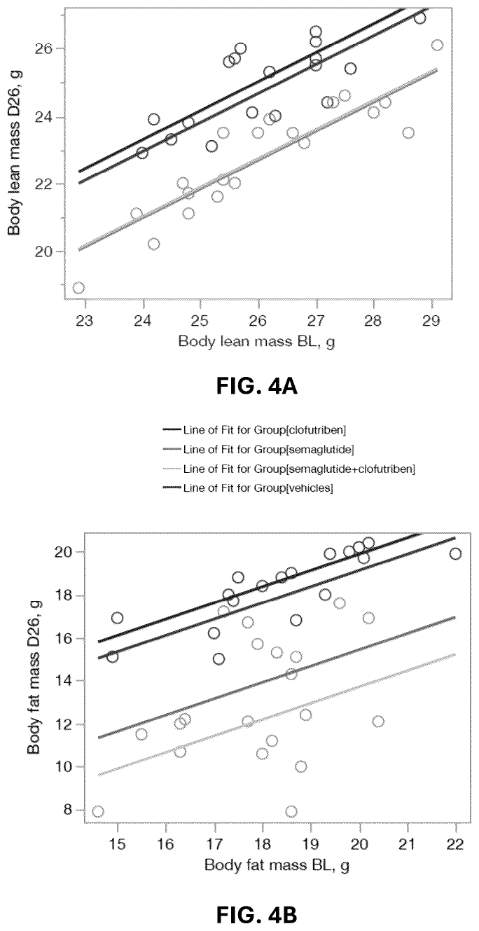

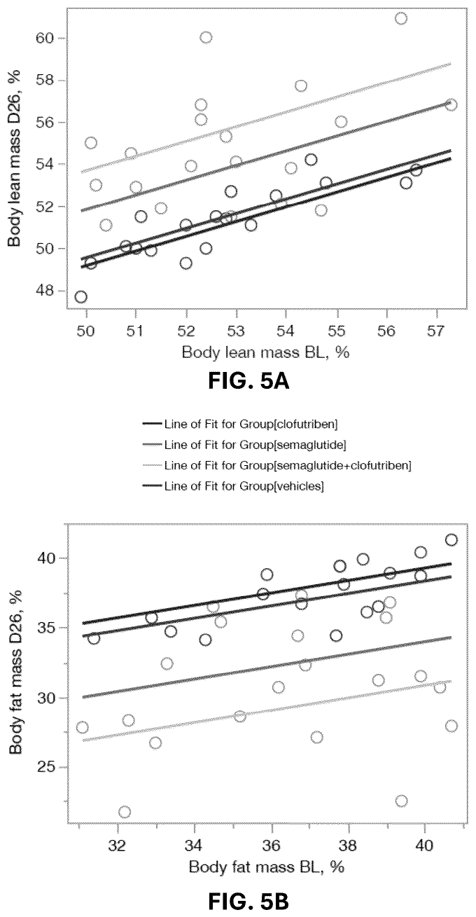

FIG. 4A and Tables 4 and 5 depict end-of-study (Study Day 26) body lean mass absolute composition to treatment and baseline value in DIO mice treated with clofutriben and semaglutide.

TABLE 4

| | Least | Std | | | | |
|---|---|---|---|---|---|---|
| Level | Sq Mean | Error | 20 | 22 | 24 | 26 |

| | | | |
|---|---|---|---|
| clofutriben | 25.0727 | 0.2697 | [symbol] |

| | | | |
|---|---|---|---|
| semaglutide | 22.7499 | 0.2561 | [symbol] |
| semaglutide + clofutriben | 22.8051 | 0.2561 | [symbol] |

| | | | |
|---|---|---|---|
| vehicles | 24.7218 | 0.2697 | [symbol] |

TABLE 5

| | | | Adjusted | Adjusted | |
|---|---|---|---|---|---|
| Group | −Group | Difference | Lower 95% | Upper 95% | t Ratio |
| clofutriben | semaglutide | 2.322815 | 1.40591 | 3.239717 | 6.25 |
| semaglutide + clofutriben | semaglutide | 0.055166 | −0.83860 | 0.948936 | 0.15 |
| vehicles | semaglutide | 1.971904 | 1.05519 | 2.888615 | 5.30 |

FIG. 4B and Tables 6 and 7 depict end-of-study (Study Day 26) body fat mass absolute composition to treatment and baseline value in DIO mice treated with clofutriben and semaglutide.

TABLE 6

| | Least | Std | | | | |
|---|---|---|---|---|---|---|
| Level | Sq Mean | Error | 8 | 12 | 16 | 20 |
| clofutriben | 18.4542 | 0.6952 | | | [symbol] | |
| semaglutide | 14.0027 | 0.6563 | | [symbol] | | |
| semaglutide + clofutriben | 12.2800 | 0.6559 | | [symbol] | | |

| | | | |
|---|---|---|---|
| vehicles | 17.6984 | 0.6901 | [symbol] |

TABLE 7

| | | | Adjusted | Adjusted | |
|---|---|---|---|---|---|
| Group | −Group | Difference | Lower 95% | Upper 95% | t Ratio |
| clofutriben | semaglutide | 4.45152 | 2.08315 | 6.819891 | 4.63 |
| semaglutide + clofutriben | semaglutide | −1.72274 | −4.00467 | 0.559195 | −1.86 |
| vehicles | semaglutide | 3.69573 | 1.34514 | 6.046312 | 3.88 |

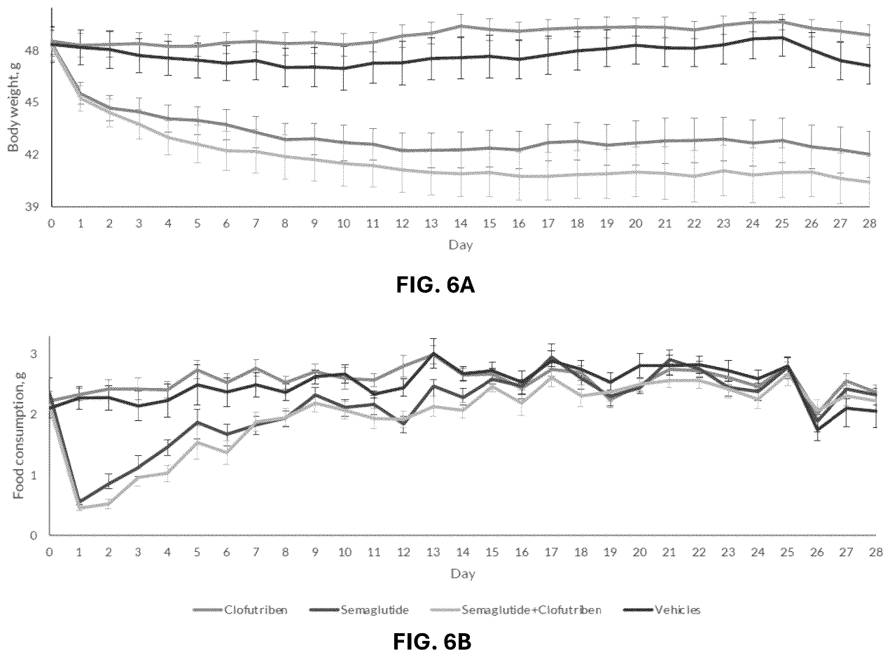

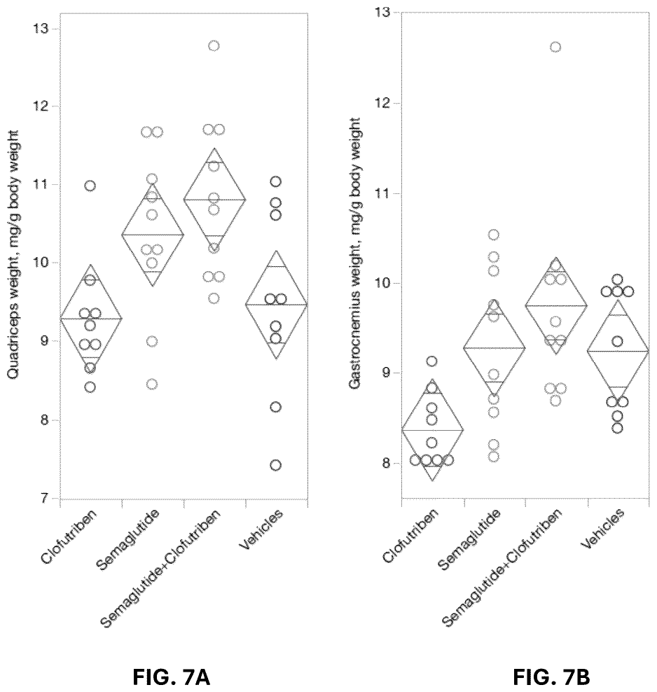

FIG. 5A and Tables 8 and 9 depict end-of-study (Study Day 26) body lean mass percent composition to treatment and baseline value in DIO mice treated with clofutriben and semaglutide.

TABLE 8

| | Least | Std | | | | |
|---|---|---|---|---|---|---|
| Level | Sq Mean | Error | 48 | 52 | 56 | 60 |
| clofutriben | 51.1149 | 0.6196 | | [symbol] | | |
| semaglutide | 53.7722 | 0.5881 | | [symbol] | | |
| semaglutide + clofutriben | 55.6182 | 0.5864 | | | [symbol] | |
| vehicles | 51.4957 | 0.6179 | | [symbol] | | |

TABLE 9

| | | | Adjusted | Adjusted | |
|---|---|---|---|---|---|
| Group | −Group | Difference | Lower 95% | Upper 95% | t Ratio |
| clofutriben | semaglutide | −2.65734 | −4.77009 | −0.54459 | −3.10 |
| semaglutide + clofutriben | semaglutide | 1.84595 | −0.20412 | 3.89603 | 2.22 |
| vehicles | semaglutide | −2.27654 | −4.37709 | −0.17598 | −2.67 |

FIG. 5B and Tables 10 and 11 depict end-of-study (Study Day 26) body fat mass percent composition to treatment and baseline value in DIO mice treated with clofutriben and semaglutide.

TABLE 10

| | Least | Std | | | | |
|---|---|---|---|---|---|---|
| Level | Sq Mean | Error | 25 | 30 | 35 | 40 |
| clofutriben | 37.7853 | 1.113 | | | [symbol] | |
| semaglutide | 32.4818 | 1.052 | | [symbol] | | |
| semaglutide + clofutriben | 29.3582 | 1.051 | | [symbol] | | |

| | | | |
|---|---|---|---|
| vehicles | 36.8591 | 1.107 | [symbol] |

TABLE 11

| | | | Adjusted | Adjusted | |
|---|---|---|---|---|---|
| Group | −Group | Difference | Lower 95% | Upper 95% | t Ratio |
| clofutriben | semaglutide | 5.30349 | 1.51273 | 9.094251 | 3.45 |
| semaglutide + clofutriben | semaglutide | −3.12358 | −6.78383 | 0.536663 | −2.10 |
| vehicles | semaglutide | 4.37735 | 0.60939 | 8.145305 | 2.86 |

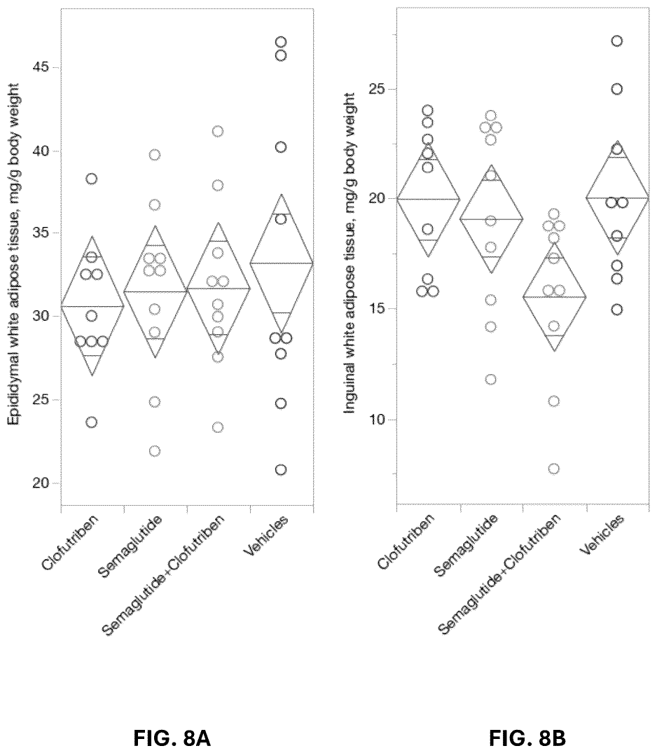

FIG. 6A depicts daily body weight by treatment in DIO mice treated with clofutriben and semaglutide. FIG. 6B depicts food consumption by treatment in DIO mice treated with clofutriben and semaglutide.

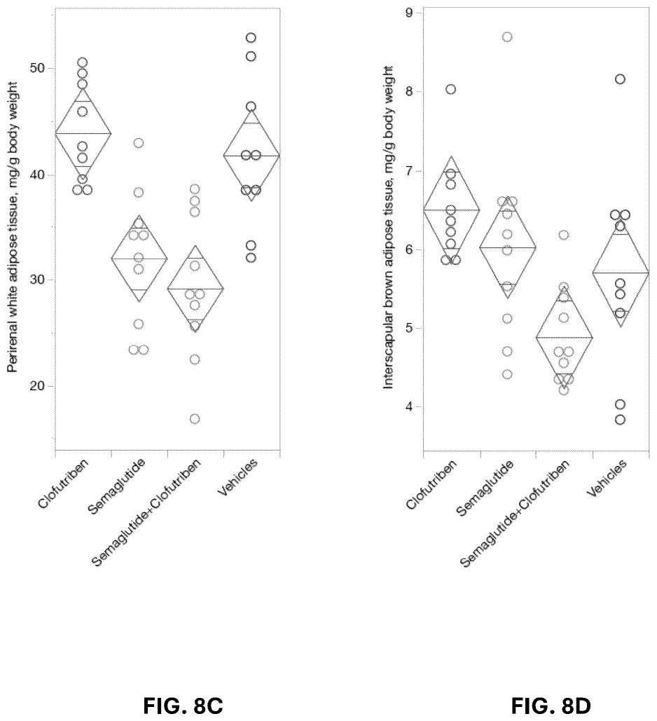

FIG. 7A and FIG. 7B depict post-mortem muscle weight by treatment in DIO mice treated with clofutriben and semaglutide.

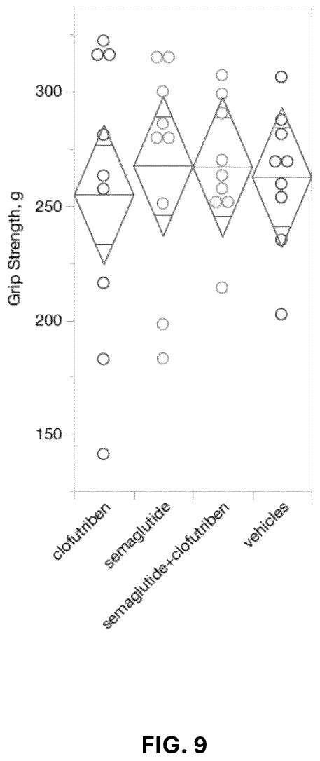

FIG. 8A depicts post-mortem epididymal white adipose tissue depot weights by treatment in DIO mice treated with clofutriben and semaglutide. FIG. 8B depicts post-mortem inguinal white adipose tissue depot weights by treatment in DIO mice treated with clofutriben and semaglutide. FIG. 8C depicts post-mortem perirenal white adipose tissue depot weights by treatment in DIO mice treated with clofutriben and semaglutide. FIG. 8D depicts post-mortem interscapular brown adipose tissue depot weights by treatment in DIO mice treated with clofutriben and semaglutide.

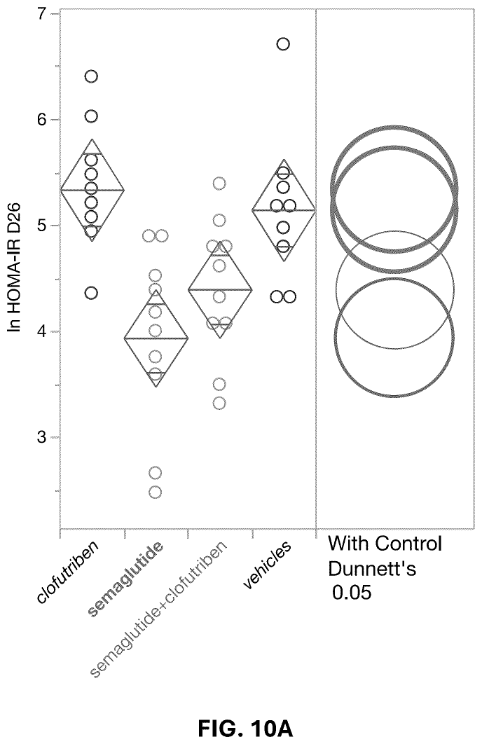

FIG. 9 depicts grip strength by treatment in DIO mice treated with clofutriben and semaglutide. Baseline was not a significant covariate for grip strength.

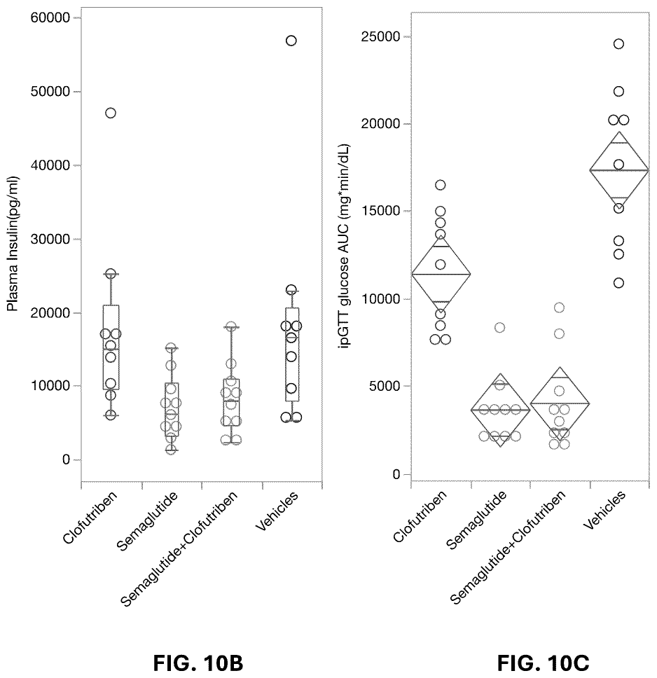

FIG. 10A depicts Homeostatic Model Assessment of Insulin Resistance (HOMA-IR) by treatment in DIO mice treated with clofutriben and semaglutide. HOMA-IR is the product of fasting glucose*fasting insulin. HOMA-IR was increased in DIO mouse relative to controls. Baseline was not a significant covariate for HOMA-IR. FIG. 10B depicts plasma by treatment in DIO mice treated with clofutriben and semaglutide. Fasting insulin data did not follow a normal distribution. FIG. 10C depicts intraperitoneal glucose tolerance test area under the time-concentration curve for post-prandial glucose (ipGTT glucose AUC) by treatment in DIO mice treated with clofutriben and semaglutide. IpGTT glucose AUC was calculated using the trapezoidal rule. Baseline was not a significant covariate for these parameters.

Clofutriben by itself showed no effect, compared to vehicle, on body weight, body composition, food consumption, quadriceps weight, adipose depot weights, grip strength, HOMA-IR, or fasting plasma insulin. Clofutriben was associated with reduced gastrocnemius weight and improved response (lower glucose area under the time-concentration curve) to ipGTT.

Semaglutide by itself showed no effect, compared to vehicle, on muscle weights, epididymal, inguinal, and intrascapacular adipose depot weights, or grip strength. Semaglutide was associated with decreased body weight, absolute body lean and fat composition, body weight-normalized fat composition, food consumption, perirenal adipose depot weight, HOMA-IR, fasting plasma insulin, and ipGTT response. Semaglutide was associated with increased body weight-normalized lean composition. Animals who received semaglutide showed reduced food consumption during the first two weeks of the study but not during the third and fourth weeks. The pattern of weight loss paralleled food consumption, with weight loss observed mostly in the first two weeks.

Compared to semaglutide monotherapy, co-administration of clofutriben and sibutramine led to further improvement on weight and body composition. The synergistic effect of clofutriben appeared to be mainly an enhancement of fat loss, leading to further increase of lean mass as a percentage of body weight but not an absolute increase of lean mass. Food consumption was undifferentiated. The benefit of clofutriben co-administration was observed as decreased inguinal and intrascapular, but not epididymal or perirenal, adipose depot weights. Muscle masses were similar between semaglutide and semaglutide+clofutriben groups. Clofutriben co-administration did not show additive benefit on glycemic control parameters.

DIO mouse has established face and translational validity as a model for patients with obesity. The results of this study suggest that such patients may gain additional benefit by co-administration of clofutriben with semaglutide. One skilled in the art would expect that the results of this study are generalizable to all HSD-1 inhibitors and all GLP-1 antagonists.

### CLAUSES

Clause 1. A method of administering a GLP-1 receptor agonist to a subject in need thereof, comprising: administering an effective amount of the GLP-1 receptor agonist; and administering simultaneously, separately, or sequentially in combination an effective amount of a HSD-1 inhibitor.

Clause 2. A method of treating obesity in a subject in need thereof, comprising: administering an effective amount of a GLP-1 receptor agonist; and administering simultaneously, separately, or sequentially in combination an effective amount of a HSD-1 inhibitor.

Clause 3. A method of inducing weight loss with maintenance of lean muscle mass in a subject in need of weight loss, comprising: administering an effective amount of a GLP-1 receptor agonist; and administering simultaneously, separately, or sequentially in combination an effective amount of a HSD-1 inhibitor so as to maintain lean muscle mass while inducing fat and weight loss in the subject.

Clause 4. A method of increasing total weight loss caused by administration of a pre-determined amount of a GLP-1 receptor agonist to a subject in need thereof, the method comprising: co-administering to a subject in need thereof, simultaneously, separately, or sequentially, an effective amount of a HSD-1 inhibitor, to increase total weight loss in the subject relative to weight loss caused by administration of a pre-determined amount of a GLP-1 receptor agonist alone.

Clause 5. A method of treating or preventing further muscle mass decrease caused by administration of a GLP-1 receptor agonist to a subject in need thereof, the method comprising: adding an effective dose of an effective amount of a HSD-1 inhibitor to a GLP-1 receptor agonist treatment regimen of a subject in need thereof, to treat or prevent further lean muscle mass decrease in the subject.

Clause 6. A method of improving glycemic control in a subject in need thereof, comprising: administering an effective amount of a GLP-1 receptor agonist; and administering simultaneously, separately, or sequentially in combination an effective amount of a HSD-1 inhibitor.

Clause 7. A method of reducing the risk of major adverse cardiovascular events in a subject with type 2 diabetes mellitus in need thereof, comprising: administering an effective amount of a GLP-1 receptor agonist; and administering simultaneously, separately, or sequentially in combination an effective amount of a HSD-1 inhibitor.

Clause 8. The method of any one of the preceding clauses, wherein the HSD-1 inhibitor is a pseudo-irreversible inhibitor.

Clause 9. The method of any one of the preceding clauses, wherein the HSD-1 inhibitor is clofutriben.

Clause 10. The method of any one of the preceding clauses, wherein the GLP-1 receptor agonist is administered orally.

Clause 11. The method of any one of clauses 1 to 9, wherein the GLP-1 receptor agonist is administered parenterally.

Clause 12. The method of any one of the preceding clauses, wherein the HSD-1 inhibitor is administered orally.

Clause 13. The method of any one of clauses 1 to 11, wherein the HSD-1 inhibitor is administered parenterally.

Clause 14. The method of any one of the preceding clauses, wherein the GLP-1 receptor agonist is selected from albiglutide, dulaglutide, exenatide, liraglutide, lixisenatide, semaglutide, and tirzepatide.

Clause 15. The method of any one of the preceding clauses, wherein, prior to administration of the HSD-1 inhibitor, the subject exhibits cortisol excess.

Clause 16. The method of clause 15, wherein, prior to administration of the HSD-1 inhibitor, the subject exhibits cortisol non-suppression based on an overnight dexamethasone suppression test.

Clause 17. The method of clause 15, wherein, prior to administration of the HSD-1 inhibitor, the subject exhibits urinary free cortisol above a reference range.

Clause 18. The method of clause 17, wherein the reference range is between 16 and 100 micrograms per 24 hours.

Clause 19. The method of clause 18, wherein the reference range is less than 60 micrograms per 24 hours.

Clause 20. The method of clause 18, wherein the reference range is less than 45 micrograms per 24 hours.

Clause 21. The method of clause 15, wherein, prior to administration of the HSD-1 inhibitor, the subject exhibits nighttime salivary cortisol above a reference range.

Clause 22. The method of clause 21, wherein the reference range for late night salivary cortisol is between 0.97 and 1.45 micrograms per liter.

Clause 23. The method of clause 22, wherein the reference range is less than 1.00 micrograms per liter.

Clause 24. The method of clause 15, wherein, prior to administration of the HSD-1 inhibitor, the subject exhibits an elevated urinary HSD-1 ratio.

Clause 25. The method of clause 15, wherein the subject has been diagnosed with endogenous Cushing's syndrome.

Clause 26. The method of clause 15, wherein the subject has been diagnosed with autonomous cortisol secretion.

Clause 27. The method of any one of clauses 1 to 14, wherein the subject is being chronically administered one or more glucocorticoid medicines.

Clause 28. The method of any one of clauses 1 to 14, wherein the subject has an adrenal tumor.

Clause 29. The method of any one of the preceding clauses, wherein, prior to administration of the HSD-1 inhibitor, the subject is at risk of experiencing muscle loss associated with GLP-1 receptor agonist administration.

Clause 30. The method of clause 29, wherein the subject has a sedentary lifestyle, osteopenia or osteoporosis, poor nutrition, or is at an advanced age.

Clause 31. The method of clause 29 or 30, wherein the subject is unable to engage in strength and resistance training.

Clause 32. The method of clause 29 or 30, wherein the subject has a muscle-wasting condition such as chronic kidney disease.

Clause 33. The method of any one of the preceding clauses, wherein the HSD-1 inhibitor and the GLP-1 receptor agonist are administered simultaneously.

Clause 34. The method of any one of clauses 1 to 32, wherein the HSD-1 inhibitor and the GLP-1 receptor agonist are administered separately.

Clause 35. The method of any one of clauses 1 to 32, wherein the HSD-1 inhibitor and the GLP-1 receptor agonist are administered separately and sequentially.

Clause 36. The method of any one of clauses 1 to 35, wherein the HSD-1 inhibitor is administered in a single daily dose of from about 0.2 mg to about 40 mg.

Clause 37. The method of clause 36, wherein the dose is from about 0.2 mg to about 20 mg.

Clause 38. The method of clause 36, wherein the dose is from about 1 mg to about 10 mg.

Clause 39. The method of any one of clauses 1 to 38, wherein the GLP-1 receptor agonist is administered once weekly.

Clause 40. A pharmaceutical composition comprising: a GLP-1 receptor agonist, a HSD-1 inhibitor, and optionally a pharmaceutically acceptable excipient.

Clause 41. A kit comprising: a first container and a second container, the first container comprising a GLP-1 receptor agonist, and the second container comprising a HSD-1 inhibitor.

The various embodiments described above can be combined to provide further embodiments. All of the U.S. patents, U.S. patent application publications, U.S. patent applications, foreign patents, foreign patent applications and non-patent publications referred to in this specification or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of administering a GLP-1 receptor agonist to a subject in need thereof, comprising:
administering an effective amount of the GLP-1 receptor agonist; and
administering simultaneously, separately, or sequentially in combination an effective amount of a HSD-1 inhibitor.

2. The method of claim 1, wherein the administering treats obesity in the subject in need thereof.

3. The method of claim 1, wherein the administering induces weight loss with maintenance of lean muscle mass in the subject.

4. The method of claim 1, wherein the administering improves glycemic control in the subject in need thereof.

5. The method of claim 1, wherein the administering reduces the risk of major adverse cardiovascular events in the subject, wherein the subject has type 2 diabetes mellitus.

6. The method of claim 1, wherein the HSD-1 inhibitor is a pseudo-irreversible inhibitor.

7. The method of claim 1, wherein the HSD-1 inhibitor is clofutriben.

8. The method of claim 1, wherein the GLP-1 receptor agonist is administered orally or parenterally.

9. The method of claim 1, wherein the HSD-1 inhibitor is administered orally or parenterally.

10. The method of claim 1, wherein the GLP-1 receptor agonist is selected from albiglutide, dulaglutide, exenatide, liraglutide, lixisenatide, semaglutide, and tirzepatide.

11. The method of claim 1, wherein, prior to administration of the HSD-1 inhibitor, the subject exhibits cortisol excess.

12. The method of claim 11, wherein, prior to administration of the HSD-1 inhibitor, the subject exhibits cortisol non-suppression based on an overnight dexamethasone suppression test.

13. The method of claim 11, wherein, prior to administration of the HSD-1 inhibitor, the subject exhibits urinary free cortisol between 16 and 100 micrograms per 24 hours.

14. The method of claim 13, wherein the urinary free cortisol is less than 60 micrograms per 24 hours.

15. The method of claim 13, wherein the urinary free cortisol is less than 45 micrograms per 24 hours.

16. The method of claim 11, wherein, prior to administration of the HSD-1 inhibitor, the subject exhibits nighttime salivary cortisol between 0.97 and 1.45 micrograms per liter.

17. The method of claim 16, wherein the nighttime salivary cortisol is less than 1.00 micrograms per liter.

18. The method of claim 11, wherein, prior to administration of the HSD-1 inhibitor, the subject exhibits an elevated urinary HSD-1 ratio.

19. The method of claim 11, wherein the subject has been diagnosed with endogenous Cushing's syndrome.

20. The method of claim 11, wherein the subject has been diagnosed with autonomous cortisol secretion.

21. The method of claim 1, wherein the subject is being chronically administered one or more glucocorticoid medicines.

22. The method of claim 1, wherein the subject has an adrenal tumor.

23. The method of claim 1, wherein, prior to administration of the HSD-1 inhibitor, the subject is at risk of experiencing muscle loss associated with GLP-1 receptor agonist administration.

24. The method of claim 23, wherein the subject has a sedentary lifestyle, osteopenia or osteoporosis, poor nutrition, or is at an advanced age.

25. The method of claim 23, wherein the subject is unable to engage in strength and resistance training or has a muscle-wasting condition such as chronic kidney disease.

26. The method of claim 1, wherein the HSD-1 inhibitor and the GLP-1 receptor agonist are administered simultaneously.

27. The method of claim 1, wherein the HSD-1 inhibitor and the GLP-1 receptor agonist are administered separately.

28. The method of claim 1, wherein the HSD-1 inhibitor and the GLP-1 receptor agonist are administered separately and sequentially.

29. The method of claim 1, wherein the HSD-1 inhibitor is administered in a single daily dose of from about 0.2 mg to about 40 mg, about 0.2 mg to about 20 mg, or about 1 mg to about 10 mg.

30. The method of claim 1, wherein the GLP-1 receptor agonist is administered once weekly.
